(19) United States
(12) Patent Application Publication
(10) Pub. No.: US 2024/0156534 A1
(43) Pub. Date: May 16, 2024

(54) ADAPTIVE LEARNING FOR ROBOTIC ARTHROPLASTY

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Rahul Khare, Sewickley, PA (US); Riddhit Mitra, Pittsburgh, PA (US); Matthew Russell, Pittsburgh, PA (US); Astha Prasad, Pittsburgh, PA (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 18/280,920

(22) PCT Filed: Mar. 9, 2022
(86) PCT No.: PCT/US2022/019470

§ 371 Date: Sep. 7, 2023

Related U.S. Application Data
(60) Provisional application No. 63/159,157, filed on Mar. 10, 2021.

(51) Int. Cl. A61B 34/10; A61B 34/00; A61B 34/30

(52) U.S. Cl. CPC A61B 34/10; A61B 34/25; A61B 34/30; A61B 2034/108; A61B 2034/258

(57) ABSTRACT

The present disclosure describes techniques and systems to adapt an arthroplasty system to particular users based on historical arthroplasty procedures associated with the user. Furthermore, the present disclosure provides that settings for an arthroplasty system associated with a user during multiple arthroplasty procedures can be captures. A ML model can be trained to infer settings for subsequent arthroplasty procedures for the user and the arthroplasty system adapted based on the inferred settings.

### RELATED APPLICATIONS

This is a non-provisional of, and claims the benefit of the filing date of, pending U.S. provisional patent application No. 63/159,157, filed Mar. 10, 2021, entitled “Adaptive Learning for Robotic Arthroplasty”, the entirety of which application is incorporated by reference herein.

### TECHNICAL FIELD

This disclosure relates generally to computer-aided orthopedic surgery apparatuses and methods. Particularly, this disclosure relates to learning preferences during arthroplasty procedures.

### BACKGROUND

The use of computers, robotics, and imaging are increasingly used to aid orthopedic surgery. For example, computer-aided navigation and robotics systems can be used to guide orthopedic surgical procedures. As a specific example, during robotic arthroplasty procedures, various views are presented to the surgeon related to the current step in the arthroplasty procedure. Furthermore, preferences related to planning the final implant position on the bone are used in the procedure.

The various visualizations that are presented initially default to fixed viewpoints. Additionally, the initial implant position defaults to the same fixed position. However, a surgeon might prefer views other than the initial default view. Likewise, the surgeon may prefer different implant positioning than the default. To change the initial default views or the final implant position, the surgeon carries out a number of steps, such as, by using touchscreen buttons, by using foot pedals, or by using other input devices to modify the view or adjust the implant position.

Modifying the initial default views at every step in the procedure often adds a significant amount of time to the overall procedure. Furthermore, adjusting the initial implant positioning adds time to the procedure.

### BRIEF SUMMARY

Thus, it would be beneficial to adapt the default settings and/or configuration of an arthroplasty system for individual users to reduce the number of inputs the user needs to make during the procedure to both reduce time needed to complete the procedure and also reduce opportunity for errors in the procedure.

The present disclosure provides an adaptive arthroplasty system arranged to “learn” preferences, on a user of the arthroplasty system level, related to reducing inputs to the arthroplasty system during an arthroplasty procedure. Said differently, the present disclosure provides to train a machine learning (ML) model or utilize data analytics to adapt the configuration and default settings of a robotic arthroplasty system to align the default settings to a user's historical usage of the arthroplasty system.

The following examples pertain to various embodiments of the systems and methods disclosed herein for implementation of the invention.

Example 1 is a first embodiment of the invention comprising a system, the system comprising a processor, one or more machine learning models and memory storing software that, when executed by the processor, causes the system to receive, as input to the one or more machine learning models, information about an arthroplasty procedure to be performed, generate, via the one or more machine learning models, configuration and default settings for the robotic arthroplasty system, and send the configuration and default settings to the robotic arthroplasty system.

Example 2 is an extension of example 1, or any other example disclosed herein, wherein the one or more machine learning models are trained to generate the configuration and default settings of the robotic arthroplasty system based on historical usage of one or more users of the robotic arthroplasty system.

Example 3 is an extension of example 2, or any other example was herein, wherein a training dataset for the one or more machine learning models includes particular bone types, bone features, bone dimensions or other anatomical features and structures from a plurality of arthroplasty procedures.

Example 4 is an extension of example 1, or any other example disclosed herein, wherein the one or more machine learning models take as input one or more of an identification of the user, a type of procedure being performed and patient demographics.

Example 5 is an extension of example 4, or any other example disclosed herein, wherein the configuration and default settings of the robotic arthroplasty system include one or more of implant position, a selection of views depicted in a graphical user interface of the system and an order in which the selection of views is displayed.

Example 6 is an extension of example 1, or any other example disclosed herein, wherein the one or more machine learning models are trained to discriminate at least on a user-by-user basis, such that an input of different users results in generation of different configuration and default settings.

Example 7 is an extension of example 1, or any other example disclosed herein, wherein the one or more machine learning models are updated on a per-procedure basis based on inputs received from a user during each procedure.

Example 8 is an extension of example 1, or any other example disclosed herein, wherein the software further causes the system to receive, from the arthroplasty system, an indication of a value of at least one setting for a plurality of arthroplasty procedures, the plurality of arthroplasty procedures associated with a specific user of the robotic arthroplasty system and wherein the one or more machine learning models are trained based on the values of the at least one setting, to infer a default value of the at least one setting for a subsequent arthroplasty procedure associated with the specific user.

Example 9 is an extension of example 1, or any other example disclosed herein, wherein the generated configuration and default settings comprise a user-specific configuration for the robotic arthroplasty system, the user-specific configuration including indications of a default value for at least one setting and wherein the software further causes the system to update a default configuration of the robotic arthroplasty system based on the user-specific configuration.

Example 10 is an extension of example 9, or any other example disclosed herein, wherein the software further causes the system to record, during the plurality of arthroplasty procedures, an input to change a viewpoint displayed in a graphical user interface from a first viewpoint to a second viewpoint, update the one of more machine learning models with the input to change a viewpoint and set, based on an output of the one of more machine learning models, for subsequent arthroplasty procedures, the second viewpoint as the value of a first one of the at least one setting.

Example 11 is an extension of example 9, or any other example disclosed herein, wherein the software further causes the system to record, during the plurality of arthroplasty procedures, a plurality of demographic information for the patient, update the one of more machine learning models with the input to change a viewpoint and set, based on an output of the one of more machine learning models, for subsequent arthroplasty procedures, the demographics information for the patient as the value of a third one of the at least one setting.

Example 12 is an extension of example 9, or any other example disclosed herein, wherein the software further causes the system to record, during the plurality of arthroplasty procedures, an input to change an implant location from an initial location to an alternative location, update the one of more machine learning models with the input to change a viewpoint and set, based on an output of the one of more machine learning models, for subsequent arthroplasty procedures, the alternative location as the value of a second one of the at least one setting.

Example 13 is an extension of example 1, or any other example disclosed herein, wherein the software further causes the system to receive, from the arthroplasty system, an indication of a value of the at least one setting for a second plurality of arthroplasty procedures, the second plurality of arthroplasty procedures associated with a second user of the arthroplasty system and update the one or more machine learning models, based on the value of the at least one setting for the second plurality of arthroplasty procedures, to infer a default value of the at least one setting for a subsequent arthroplasty procedure associated with the second user.

Example 14 is an extension of example 13, or any other example disclosed herein, wherein the user is a surgeon and the second user is a practice group.

Example 15 is an extension of example 1, or any other example disclosed herein, wherein the system and the robotic arthroplasty system are integral.

Other technical features may be readily apparent to one skilled in the art from the following figures, descriptions, and claims.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

It is noted, the drawings are not necessarily to scale. The drawings are merely representations, not intended to portray specific parameters of the disclosure. The drawings are intended to depict example embodiments of the disclosure, and therefore are not considered as limiting in scope. In the drawings, like numbering represents like elements.

Furthermore, certain elements in some of the figures may be omitted for illustrative clarity. The cross-sectional views may be in the form of “slices”, or “near-sighted” cross-sectional views, omitting certain background lines otherwise visible in a “true” cross-sectional view, for illustrative clarity. Furthermore, for clarity, some reference numbers may be omitted in certain drawings.

FIG. 1A illustrates an example of the subject matter in accordance with one embodiment.

FIG. 1B illustrates an example of the subject matter in accordance with one embodiment.

FIG. 2A illustrates an example of the subject matter in accordance with one embodiment.

FIG. 2B illustrates an example of the subject matter in accordance with one embodiment.

FIG. 3 illustrates an example of the subject matter in accordance with one embodiment.

FIG. 4 illustrates a routine 400 in accordance with one embodiment.

FIG. 5 illustrates an example of the subject matter in accordance with one embodiment.

FIG. 6A illustrates an example of the subject matter in accordance with one embodiment.

FIG. 6B illustrates an example of the subject matter in accordance with one embodiment.

FIG. 6C illustrates an example of the subject matter in accordance with one embodiment.

FIG. 7A illustrates an example of the subject matter in accordance with one embodiment.

FIG. 7B illustrates an example of the subject matter in accordance with one embodiment.

FIG. 8 illustrates an example computer-readable storage medium 800 in accordance with one embodiment.

FIG. 9 illustrates an example of the subject matter in accordance with one embodiment.

### DETAILED DESCRIPTION

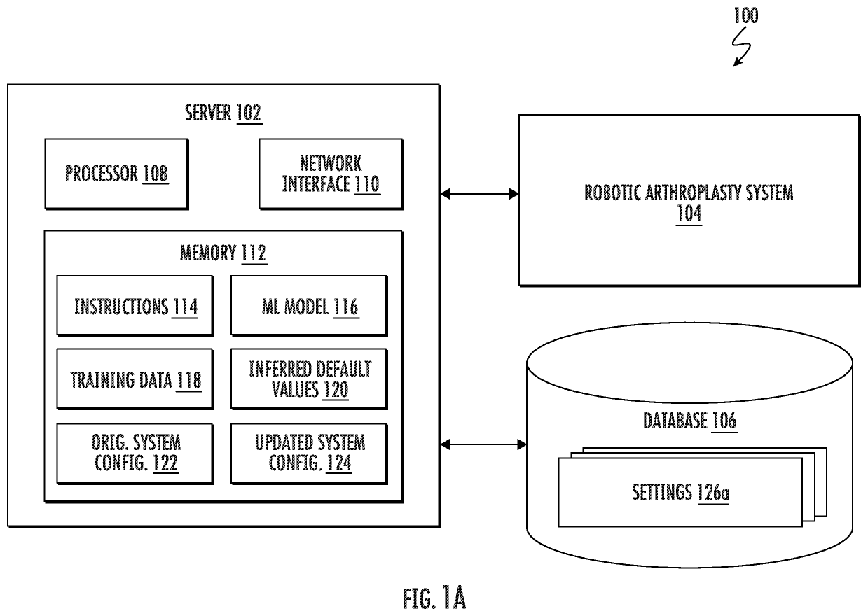

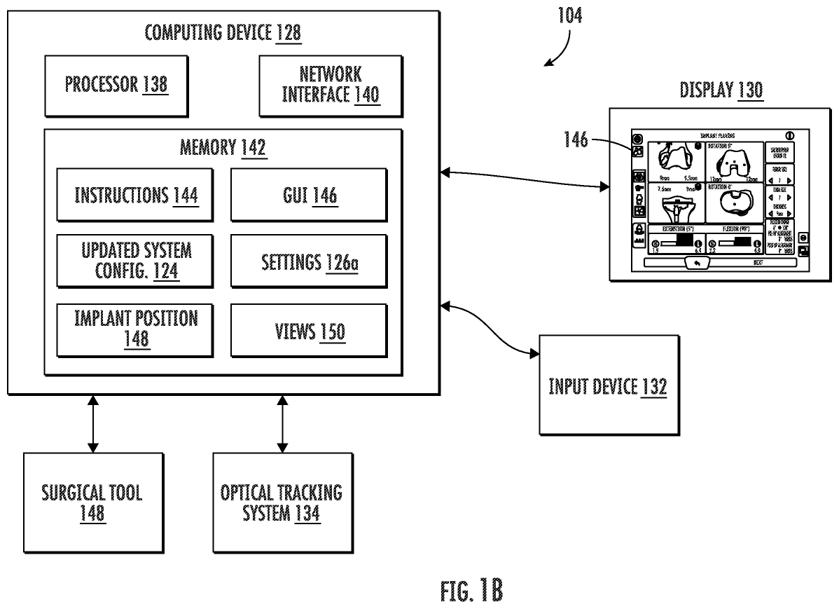

FIG. 1A and FIG. 1B illustrates an adaptive robotic surgery system 100, in accordance with non-limiting example(s) of the present disclosure. Adaptive robotic surgery system 100 includes a server 102, robotic arthroplasty system 104, and database 106. With some examples, server 102 and database 106 can be combined or implemented within robotic arthroplasty system 104. In particular, server 102 and computing device 128 could be provided by the same computing system. However, adaptive robotic surgery system 100 is depicted and described with server 102 and database 106 separate from robotic arthroplasty system 104 for clarity of presentation only.

FIG. 1A depicts details of server 102 and database 106 while FIG. 1B depicts details of robotic arthroplasty system 104. Server 102 includes processor 108, network interface 110, and memory 112. Memory 112 can include instructions 114, ML model 116, training data 118, inferred default values 120, original arthroplasty system configuration 122, and updated system configuration 124.

In general, robotic arthroplasty system 104 can be used by any of a variety of users to perform an arthroplasty procedure, such as, interpositional arthroplasty, resectional arthroplasty, resurfacing arthroplasty, mold arthroplasty, replacement arthroplasty, or the like. For example, a surgeon, a nurse, a surgical assistant, a sales representative, or other “user” could operate the robotic arthroplasty system 104. It is noted that where one particular user (e.g., a surgeon) is referenced herein, other users could be substituted without departing from the scope of the disclosure. Furthermore, the user need not be physically present but could instead be remote from the operating theater. Examples are not limited in these respects. During a typical arthroplasty procedure, the surgeon plans the implant position and toggles through a number of views of the patient's joint. For example, in knee arthroplasty, the surgeon may use kinematic alignment, measured resection technique, and/or gap balancing approach to plan a well-balanced knee. These approaches might be used in isolation or in combination. However, every surgeon plans the implant placement during an arthroplasty procedure differently. For example, one surgeon may use kinematic alignment while another surgeon uses both kinematic alignment and gap balancing. These different approaches result in different adjustments to the initial implant position. As such, each surgeon will adjust the implant position differently.

Robotic arthroplasty system 104 includes computing device 128, display 130, input device 132, optical tracking system 134, and surgical tool 136. In order to set the implant position as desired (e.g., based on the approach the surgeon prefers, or the like) the surgeon will need to adjust the position from the default using input device 132. Likewise, during an arthroplasty procedure, a surgeon often adjusts views (e.g., GUI 146, or the like) displayed on display 130 to suit personal preferences using input device 132. Views displayed in GUI 146 on display 130 can be a number of different views of the joint (e.g., from different angles, cut away views, alignment views, with the implant positioned, etc.) Input device 132 can be a foot pedal, a keyboard, a joystick, a touch screen, or the like. As can be appreciated, adjusting the implant position introduces opportunity for errors as well as takes time. Likewise, adjusting the views depicted in GUI 146 takes time. These are all undesirable in a surgical procedure.

Adaptive robotic surgery system 100 provides to adaptively adjust the configuration and/or settings of robotic arthroplasty system 104 such that the defaults (e.g., implant position, views depicted in GUI 146, or the like) are specific to the surgeon using the tool. It is noted that this is not as trivial as merely specifying preferences for robotic arthroplasty system 104. For example, multiple surgeons use the same robotic arthroplasty system 104, as such, the preferences would need to be continually changed for each user. Furthermore, surgeons often use different approaches to set the implant position, look at different views, or look at views in a specific order depending on numerous factors (e.g., the particular procedure, the particular patient, etc.). As such, merely setting “preferences” is often insufficient to yield the savings in time and error protecting mechanisms with which the present disclosure provides.

During operation, processor 108 can execute instructions 114 to receive indications of settings 126a from robotic arthroplasty system 104 and store indications of the settings 126a to database 106. As used herein, settings 126a can be settings such as the initial implant position, the default views for GUI 146, the order of views to display in GUI 146 as the procedure progresses, or the like. Likewise, during operation, processor 138 can execute instructions 144 to record and/or capture settings 126a and communicate (e.g., via network interface 140 and network interface 110, or the like) the settings 126a to server 102.

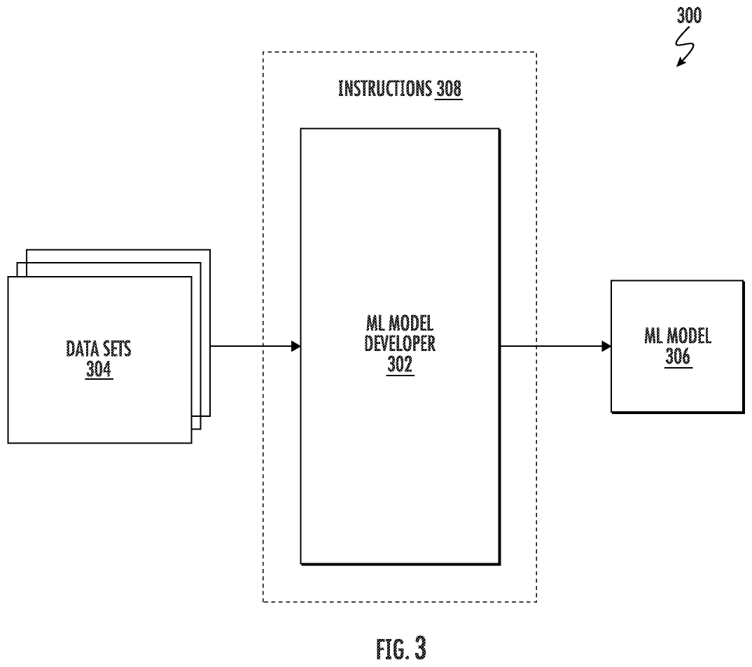

Server 102 can be arranged to store, in database 106, settings 126a for multiple users (e.g., individual surgeons, particular clinics or practice groups, etc.) over multiple arthroplasty procedures. After a sufficient (e.g., depending on the ML model, or the like) number of arthroplasty procedures have settings 126a archived in database 106, server 102 can be arranged to generate training data 118 from settings 126a and train ML model 116 using training data 118. An example of this is given later (e.g., refer to FIG. 3).

Additionally, processor 108 can execute instructions 114 to generate an inference based on ML model 116 for particular users of robotic arthroplasty system 104 (e.g., individual surgeons, practice groups, clinics, or the like). For example, processor 108 can execute instructions 114 and/or ML model 116 to generate inferred default values 120. With some examples, ML model 116 can be a classification model, a decision tree model, a dimensionality reduction model, or the like. Furthermore, with some examples, ML model 116 can be an unsupervised learning model, a supervised learning model, or a semi-supervised learning model. Examples are not limited in this context. However, as a specific example, ML model 116 can be classification model, implemented by a neural network, and arranged to classify inputs (e.g., surgeon, procedure type, patient demographic, etc.) to particular outputs (e.g., default implant position, default views, procedure viewing order, etc.). Said differently, ML model 116 can be classification model arranged to generate inferred default values 120, where the inferred default values 120 are default implant position, default views, procedure viewing order, etc.

Processor 108 can execute instructions 114 to generate an updated system configuration 124 from an original arthroplasty system configuration 122 and the inferred default values 120. In some examples, original arthroplasty system configuration 122 and updated system configuration 124 can be an information element, data structure, or other data comprising indications of the default values described herein. Processor 108 can execute instructions 114 to send updated system configuration 124 to robotic arthroplasty system 104 and/or otherwise configure, program, or signal to robotic arthroplasty system 104 to use the default values indicated by updated system configuration 124.

Additionally, during operation, processor 138 can execute instructions 144 to receive updated system configuration 124 from server 102 and to apply or otherwise configure robotic arthroplasty system 104 based on updated system configuration 124. Furthermore, processor 138 can execute instructions 144 to determine implant position 148 and views 150 from updated system configuration 124. Further still, processor 138 can execute instructions 144 to generate GUI 146 to include representation and/or depictions of initial implant position 148 and views 150.

Server 102 and computing device 128 can be any of a variety of computing devices. In some embodiments, these devices can be incorporated into and/or implemented by a console of robotic arthroplasty tool, such as, robotic arthroplasty system 104. With some embodiments, server 102 can be a workstation or server communicatively coupled to computing device 128 and/or robotic arthroplasty system 104. With still other embodiments, server 102 can be provided by a cloud-based computing device, such as, by a computing as a service system accessibly over a network (e.g., the Internet, an intranet, a wide area network, or the like).

Database 106 can be any of a variety of memory storage devices arranged to store indications of settings 126a. For example, database 106 can be a non-transitory memory storage array (e.g., hard disk drives, solid-state drives, or the like) with a file structure and data storage archiving system arranged to store indications of settings 126a.

Processor 108 and processor 138 may include circuitry or processor logic, such as, for example, any of a variety of commercial processors. In some examples, processor 108 and/or processor 138 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 108 and/or processor 138 may include graphics processing portions and may include dedicated memory, multiple-threaded processing and/or some other parallel processing capability. In some examples, the processor 108 may be an application specific integrated circuit (ASIC) or a field programmable integrated circuit (FPGA).

Memory 112 and memory 142 may include logic, a portion of which includes arrays of integrated circuits, forming non-volatile memory to persistently store data or a combination of non-volatile memory and volatile memory. It is to be appreciated, that the memory 112 and/or memory 142 may be based on any of a variety of technologies. In particular, the arrays of integrated circuits included in memory 112 and/or memory 142 may be arranged to form one or more types of memory, such as, for example, dynamic random access memory (DRAM), NAND memory, NOR memory, or the like.

Network interface 110 and network interface 140 can include logic and/or features to support a communication interface. For example, network interface 110 and/or network interface 140 may include one or more interfaces that operate according to various communication protocols or standards to communicate over direct or network communication links. Direct communications may occur via use of communication protocols or standards described in one or more industry standards (including progenies and variants). For example, network interface 110 and/or network interface 140 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like. Additionally, network interface 110 and/or network interface 140 can include logic and/or features to enable communication over a variety of wired or wireless network standards (e.g., 802.11 communication standards). For example, network interface 110 and/or network interface 140 may be arranged to support wired communication protocols or standards, such as, Ethernet, or the like. As another example, network interface 110 and/or network interface 140 may be arranged to support wireless communication protocols or standards, such as, for example, Wi-Fi, Bluetooth, ZigBee, LTE, 5G, or the like.

As noted, input device 132 can be a foot pedal, a keyboard, a joystick, a touch screen, or the like. In other examples, input device 132 can be incorporated into display 130 and/or surgical tool 136. As a specific example, display 130 can be a touch screen display and/or surgical tool 136 can include a hand piece with trigger or toggle switches arranged as input device 132. As a specific example, surgical tool 136 can be an orthopedic cutting tool (e.g., a bur, a drill, a reciprocating saw, or the like) for cutting and surfacing the bone.

Generally speaking, optical tracking system 134 is a 3D localization can be technology that could be used to track active or passive markers in space. These markers can be fixated on tracking frames to objects that need to be localized in 3D space such as bone screws (that are, in turn, drilled into bones that need to be localized), point probes, cutting tools etc. The features and functions described herein with respect to optical tracking system 134 could be implemented in commercially available optical tracking systems could be, such as, infrared-based tracking system (e.g., systems (e.g., NDI Polaris Vega, Atracsys FusionTrack 500, or the like) or video-tracking systems (e.g., Microntracker from ClaroNav, or the like).

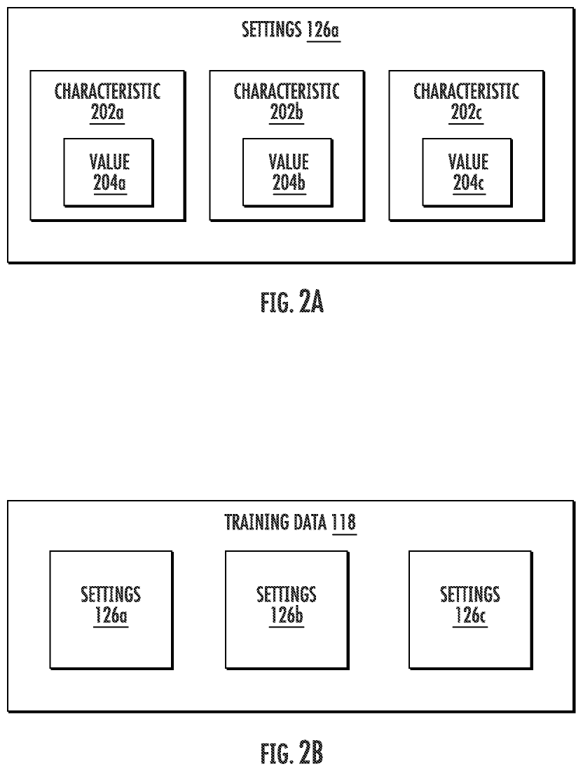

FIG. 2A illustrates exemplary settings 126a according to one or more embodiments described hereby. In the illustrated embodiment, settings 126a includes a number (e.g., one or more) of characteristic 202a, characteristic 202b, and characteristic 202c. Each of characteristic 202a, characteristic 202b, and characteristic 202c includes at least one value. For example, value 204a, value 204b, and value 204c are depicted. Generally, a characteristic may represent an option of robotic arthroplasty system 104 from an arthroplasty procedure. As a specific example, characteristic 202a may include implant position and value 204a may include the final implant position; characteristic 202b may include views selectable by the user while value 204b may include the views selected; characteristic 202c may include the order of views selected by the user and value 204c may include the actual ordering of views. Furthermore, settings 126a can also include indications of the user of robotic arthroplasty system 104, demographic information for the patient, the arthroplasty procedure type, or the like.

As described herein, the present disclosure is directed towards adapting robotic arthroplasty system 104 to multiple users (e.g., surgeons, clinics, practice groups, or the like). To this end, numerous settings 126a from multiple arthroplasty procedures will be collected, for example, as described above.

Once sufficient (e.g., tens, hundreds, or the like) settings 126a are captured and archived in database 106, a training data 118 can be generated to train (or retrain as may be the case) ML model 116. FIG. 2B illustrates an exemplary training data 118 according to one or more embodiments described hereby. In the illustrated embodiment, training data 118 includes a number of settings (e.g., one or more). In particular, training data 118 includes settings 126a, settings 126b, and settings 126c. In various embodiments, training data 118 may be used to train one or more ML models. It is to be appreciated (although not depicted here) that training data 118 includes both training data and testing data. That is, some samples may be used for training ML model 116 (e.g., based on an ML model training algorithm, an adversarial training algorithm, or the like) while other samples may be used to test the inference quality of the trained ML model 116.

As further contemplated in this disclosure, models of a patient's anatomy or bone structure with which to represent in various GUIs and/or with which to plan implant positioning and provide feedback on a treatment plan are utilized by robotic surgery system 100. For example, ML model 116 can be trained based on particular bone types, bone features, bone dimensions, or other anatomical features and structure. With some examples, actual measurements of a patient's bone structure are used to generate such a model while in other examples, images (e.g., from an X-Ray, MRI, or the like) are used to morph a bone model.

Although the present disclosure is not particularly directed towards actual training methodologies of ML models, an example system is provided here for clarity of presentation and to more fully appreciate the novelty and difficulty of adapting robotic arthroplasty system 104 for individual users. FIG. 3 illustrates an exemplary operating environment 300 according to one or more embodiments described herein. Operating environment 300 may include ML model developer 302, data sets 304, and ML model 306. Note that ML model 306 can be ML model 116, can be a retained or further trained version of ML model 116, or can be an entirely different ML model. Furthermore, data sets 304 can include training data 118.

In various embodiments, ML model developer 302 may utilize one or more ML model training algorithms (e.g., backpropagation, convolution, adversarial, or the like) to train ML model 306 from data sets 304. Often, training ML model 306 is an iterative process where weights and connections within ML model 306 are adjusted to converge upon a satisfactory level of inference (e.g., output) for ML model 306. In some examples, ML model developer 302 can be incorporated in instructions 308 and executed by a processor (e.g., processor 108, processor 138, or the like).

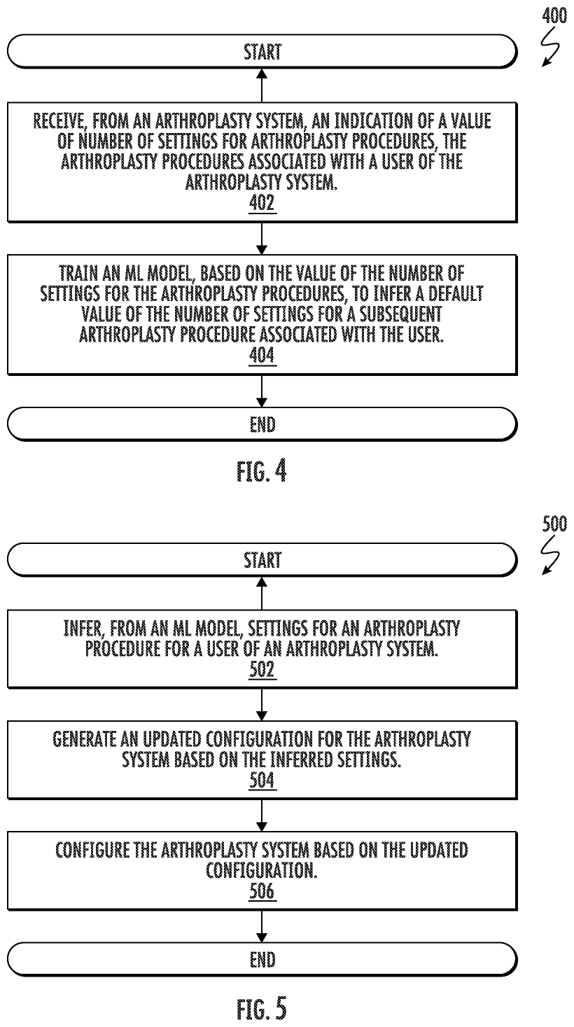

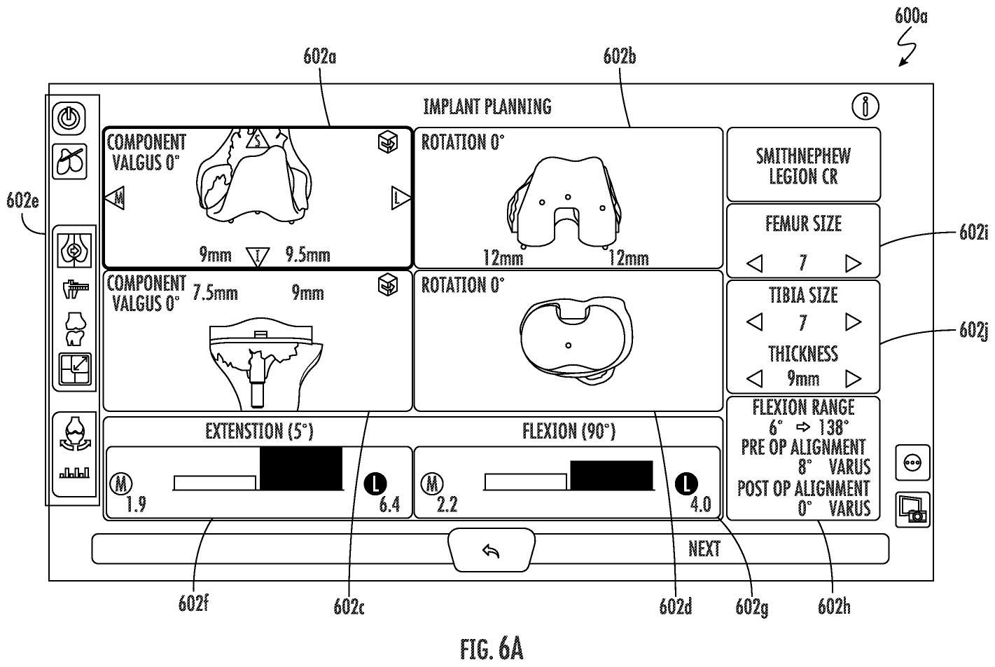

FIG. 4 illustrates a routine 400, in accordance with non-limiting example(s) of the present disclosure. Routine 400 can begin at block 402. At block 402 “receive, from an arthroplasty system, an indication of a value of number of settings for arthroplasty procedures, the arthroplasty procedures associated with a user of the arthroplasty system” settings for a number of arthroplasty procedures can be received. For example, server 102 can receive from robotic arthroplasty system 104 indications of settings 126a associated with a user of robotic arthroplasty system 104 from an arthroplasty procedure as well as settings 126b and/or settings 126c associated with the user from other arthroplasty procedures.

As a specific example, processor 138 can execute instructions 144 to record, save, or otherwise capture indications of final implant position, views selected, order of views selected, etc. during an arthroplasty procedure and store the captured indications as settings 126a. Processor 138 can further execute instructions 144 to send an information element comprising indications of the settings 126a to server 102. Likewise, processor 108 can execute instructions 114 to receive from robotic arthroplasty system 104 the information element comprising indications of settings 126a.

Routine 400 can continue to block 404 “train an ML model, based on the value of the number of settings for the arthroplasty procedures, to infer a default value of the number of settings for a subsequent arthroplasty procedure associated with the user” an ML model can be trained based on the settings received at block 402 to infer settings for subsequent arthroplasty procedures for the user. For example, server 102 can generate a training data 118, from settings 126a, settings 126b, settings 126c, etc. to train ML model 116 to generate inferred default values 120. In particular, processor 108 can execute instructions 114 (e.g., including ML model developer 302, or the like) to train ML model 116.

FIG. 5 illustrates a routine 500, in accordance with non-limiting example(s) of the present disclosure. Routine 500 can begin at block 502. At block 502 “infer, from an ML model, settings for an arthroplasty procedure for a user of an arthroplasty system” settings for an arthroplasty procedure can be inferred from an ML model. For example, processor 108 can execute instructions 114 to generate inferred default values 120 from ML model 116.

Continuing to block 504 “generate an updated configuration for the arthroplasty system based on the inferred settings” an updated configuration for the arthroplasty system can be generated, based on the inferred settings. For examples, processor 108 can execute instructions 114 to generate updated system configuration 124 from original arthroplasty system configuration 122 and inferred default values 120.

Continuing to block 506 “configure the arthroplasty system based on the updated configuration” the arthroplasty system can be configured based on the updated arthroplasty system configuration. For example, processor 108 can execute instructions 114 to send an information element comprising indications of the updated system configuration 124. Likewise, processor 138 can execute instructions 144 to receive an information element comprising indications of the updated system configuration 124 and can configure or otherwise program the robotic arthroplasty system 104 based on the updated system configuration 124.

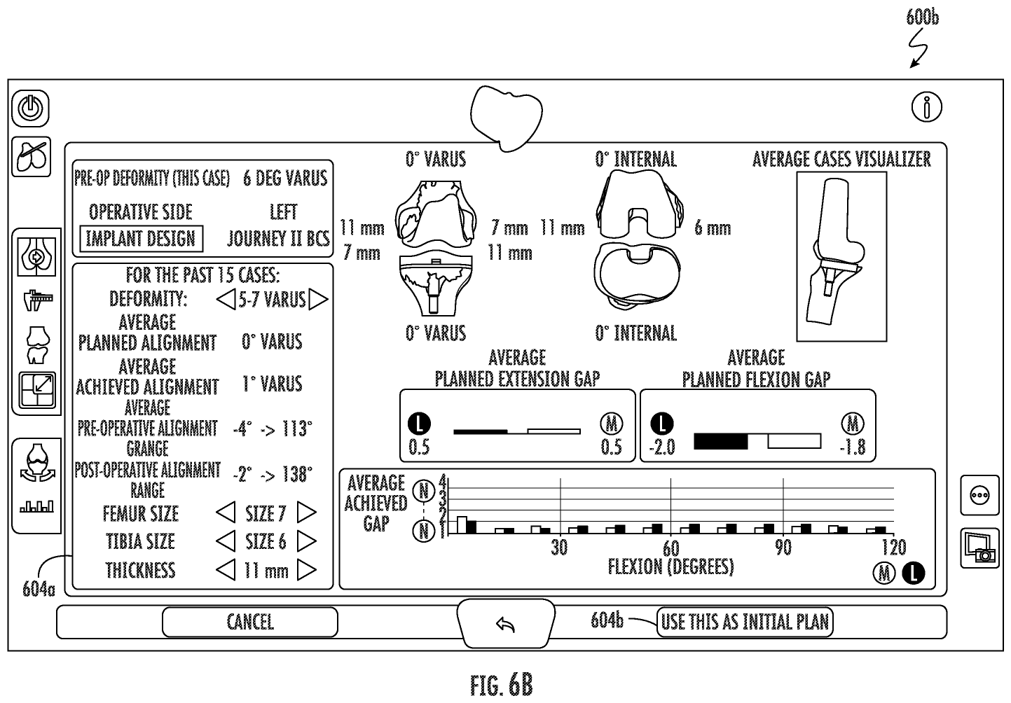

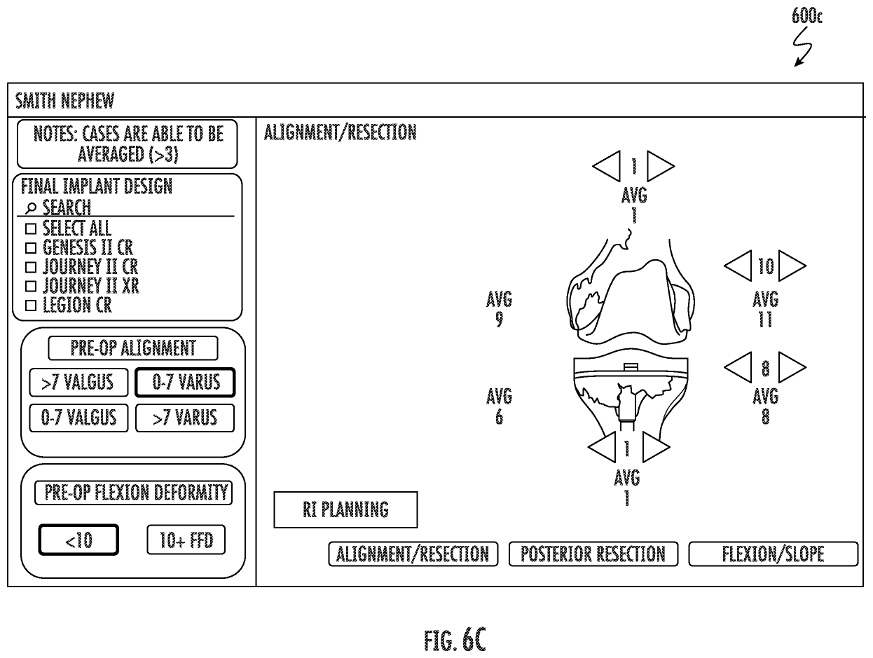

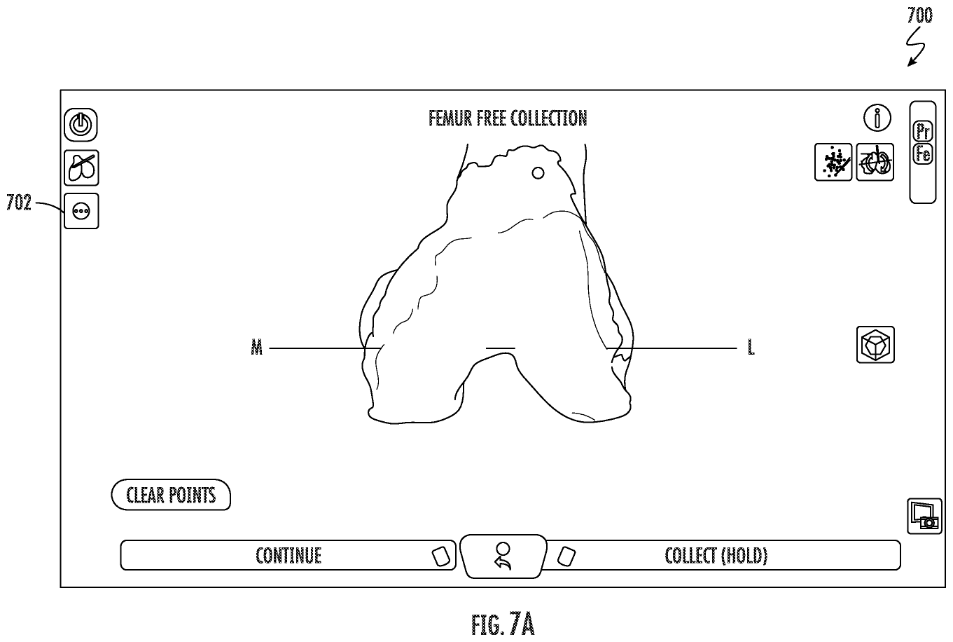

FIG. 6A illustrates an example GUI 600a, in accordance with non-limiting example(s) of the present disclosure. With some implementations, GUI 600a can be GUI 146 displayed on display 130 of adaptive robotic surgery system 100. As depicted, GUI 600a includes a number of GUI elements, such as GUI elements 602a, 602b, 602c, 602d, 602e, 602f, 602g, 602h, 602i, and 602j. In general, GUI 600a is representative of a GUI that may be generated as part of planning arthroplasty for the knee joint, and particularly for planning implant location with adaptive robotic surgery system 100. For example, GUI elements 602i and 602j depict general features or size of the femur and tibia with which the arthroplasty procedure is to be performed. GUI elements 602a, 602b, 602c, and 602d depict views of the implant to be placed during the arthroplasty procedure along with initial placement positions of the implant. Likewise, GUI elements 602f, 602g, and 602h depict mechanical behavior of the implant based on the positions reflected in the other GUI elements. In particular, GUI element 602f depicts extension of the joint while GUI element 602g depicts flexion of the joint.

In general, the implant position depicted in GUI 600a and be based on implant positions 148 generated as described herein. For example, ML model 116 can be trained to generate implant positions 148 based on database 106 including indications of implant positions for prior arthroplasty procedures performed by a particular surgeon, by surgeons in a physician group or hospital group, or based on implant positions from technical literature (e.g., medical journals, or the like). As noted, ML model 116 can be trained to infer settings, system configuration and other information relevant to an arthroplasty procedure or to a robotic arthroplasty system 104, such as, adaptive robotic surgery system 100. With some implementations, ML model 116 can be used to infer an initial treatment plan for arthroplasty surgery.

FIG. 6B illustrates an example GUI 600b, in accordance with non-limiting example(s) of the present disclosure. With some implementations, GUI 600b can be GUI 146 displayed on display 130 of adaptive robotic surgery system 100. Like GUI 600a, GUI 600b includes a number of GUI elements. It is noted that not all GUI elements of GUI 600b (or GUI 600a for that matter) are called out for purposes of clarity of presentation. However, GUI 600b can include GUI element 604a including an indication of implant positions for prior arthroplasty surgeries with which ML model 116 can be trained to infer a treatment plan from. Information related to the suggested treatment plan can be depicted in GUI 600b. Likewise, GUI 600b can provide for a user to manipulate the suggested treatment plan, use the suggested treatment plan (e.g., GUI element 604b, or the like), or cancel the suggested treatment plan.

FIG. 6C illustrates another example GUI 600c, in accordance with non-limiting example(s) of the present disclosure. Like GUI 600b of FIG. 6B, GUI 600c includes GUI elements ((not individually called out) depicting suggested implant positions for an arthroplasty procedure. With some examples, a user can select which type of implant and/or implant design is to be used and ML model 116 can generate inferences of implant positions accordingly.

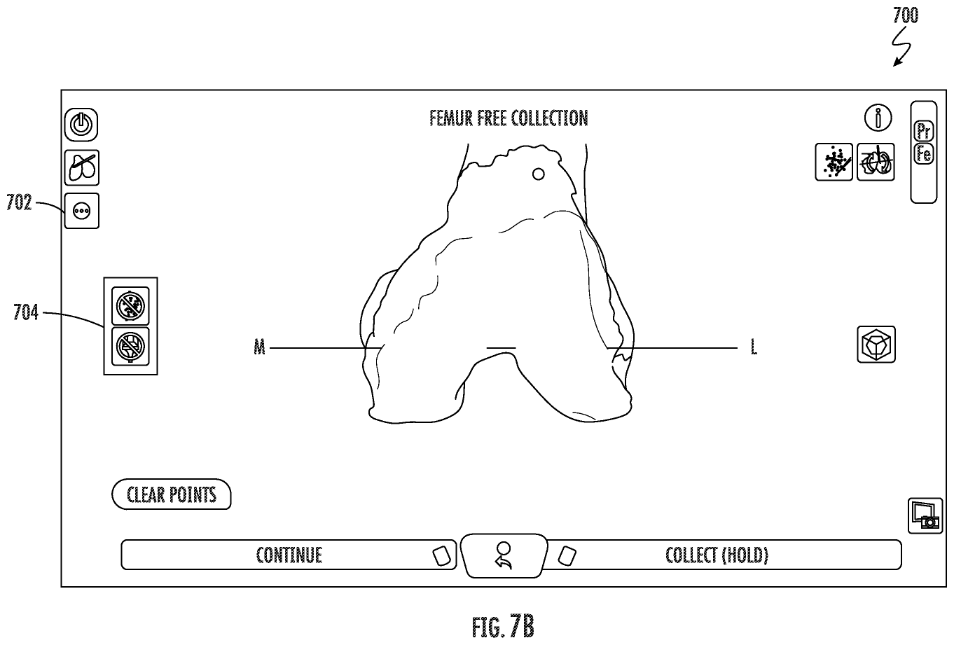

FIG. 7A and FIG. 7B depict example GUI 700, in accordance with non-limiting example(s) of the present disclosure. With some implementations, GUI 700 can be GUI 146 displayed on display 130 of adaptive robotic surgery system 100. In particular, as detailed above, ML model 116 can be trained to infer updated system configuration 124, which can include modification to default GUIs or ordering of GUIs displayed during setup and use of adaptive robotic surgery system 100. For example, some users of a robotic arthroplasty tool may not utilize or visit all possible GUIs or “screens” with which the tool can present to a user. As such, ML model 116 can generate updated system configuration 124 and from updated system configuration 124, GUI 146. GUI 700 depicts an example of a GUI that can be generated from updated system configuration 124. GUI 700 includes GUI element 702. In some examples, GUI element 702 can be an active GUI element or “tool tip” type GUI element where actions (e.g., mouse over, click, hot key press, or the like) activate the GUI element. FIG. 7A illustrates GUI 700 with 702 in the inactive state.

FIG. 7B illustrates the GUI 700 with GUI element 702 in the active state. When activated, GUI element 702 can unlock or otherwise make visible GUI element 704. Said differently, GUI element 704 can be hidden behind GUI element 702 and can be viewed or made visible when GUI element 702 is activated. In some examples, GUI element 704 can correspond to features of adaptive robotic surgery system 100 that are not used by a particular user. As such, ML model 116 can be trained to generate updated system configuration 124 for individual users, practice groups, hospitals, or the like and GUIs 146 (e.g., GUI 700, or the like) can be generated such that components of adaptive robotic surgery system 100 (e.g., setting screens, or the like) may be hidden and not presented to the user unless requested (e.g., via activating 702, or the like).

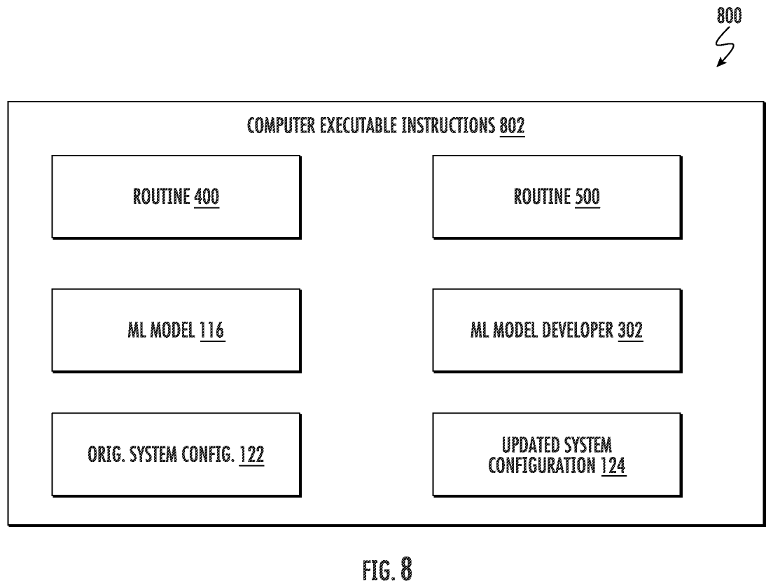

FIG. 8 illustrates computer-readable storage medium 800. Computer-readable storage medium 800 may comprise any non-transitory computer-readable storage medium or machine-readable storage medium, such as an optical, magnetic or semiconductor storage medium. In various embodiments, computer-readable storage medium 800 may comprise an article of manufacture. In some embodiments, 700 may store computer executable instructions 802 with which circuitry (e.g., processor 108, processor 138, or the like) can execute. For example, computer executable instructions 802 can include instructions to implement operations described with respect to routine 400, routine 500, ML model 116, ML model developer 302, original arthroplasty system configuration 122 and/or updated system configuration 124. Examples of computer-readable storage medium 800 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 802 may include any suitable type of code, such as source code, compiled code, interpreted code, executable code, static code, dynamic code, object-oriented code, visual code, and the like.

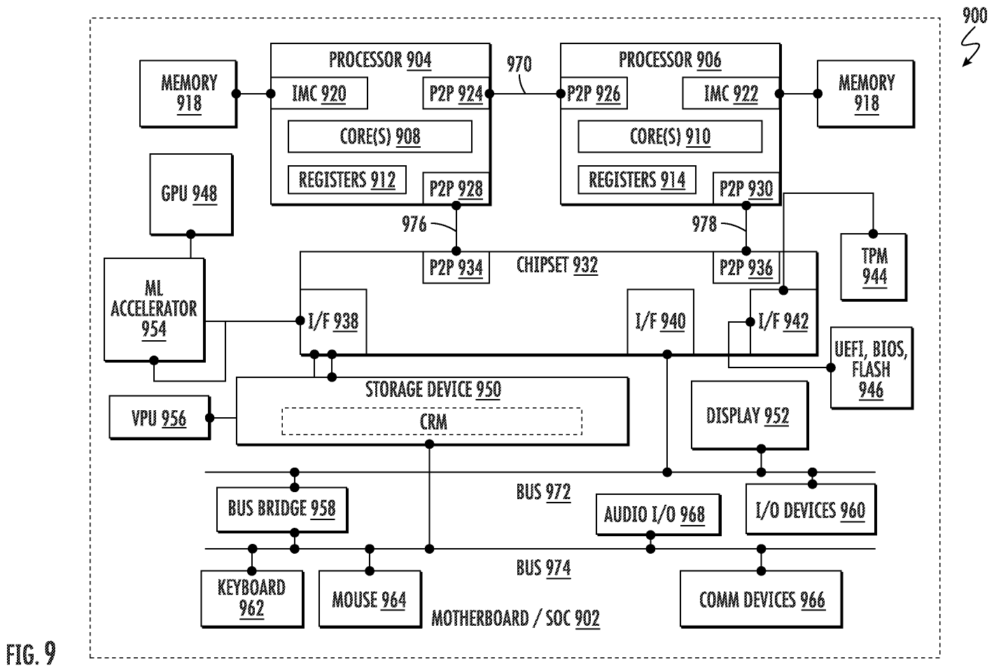

FIG. 9 illustrates an embodiment of a system 900. System 900 is a computer system with multiple processor cores such as a distributed computing system, supercomputer, high-performance computing system, computing cluster, mainframe computer, mini-computer, client-server system, personal computer (PC), workstation, server, portable computer, laptop computer, tablet computer, handheld device such as a personal digital assistant (PDA), or other device for processing, displaying, or transmitting information. Similar embodiments may comprise, e.g., entertainment devices such as a portable music player or a portable video player, a smart phone or other cellular phone, a telephone, a digital video camera, a digital still camera, an external storage device, or the like. Further embodiments implement larger scale server configurations. In other embodiments, the system 900 may have a single processor with one core or more than one processor. Note that the term “processor” refers to a processor with a single core or a processor package with multiple processor cores. In at least one embodiment, the computing system 900 is representative of the components of the adaptive robotic surgery system 100, such as server 102 and/or computing device 128. More generally, the computing system 900 is configured to implement all logic, systems, logic flows, methods, apparatuses, and functionality described herein with reference to FIG. 1A, FIG. 1B, FIG. 2A, FIG. 2B, FIG. 3, FIG. 4, FIG. 5, FIG. 6A, FIG. 6B, FIG. 6C, FIG. 7A, FIG. 7B, and FIG. 8.

As used in this application, the terms “system” and “component” and “module” are intended to refer to a computer-related entity, either hardware, a combination of hardware and software, software, or software in execution, examples of which are provided by the exemplary system 900. For example, a component can be, but is not limited to being, a process running on a processor, a processor, a hard disk drive, multiple storage drives (of optical and/or magnetic storage medium), an object, an executable, a thread of execution, a program, and/or a computer. By way of illustration, both an application running on a server and the server can be a component. One or more components can reside within a process and/or thread of execution, and a component can be localized on one computer and/or distributed between two or more computers. Further, components may be communicatively coupled to each other by various types of communications media to coordinate operations. The coordination may involve the uni-directional or bi-directional exchange of information. For instance, the components may communicate information in the form of signals communicated over the communications media. The information can be implemented as signals allocated to various signal lines. In such allocations, each message is a signal. Further embodiments, however, may alternatively employ data messages. Such data messages may be sent across various connections. Exemplary connections include parallel interfaces, serial interfaces, and bus interfaces.

As shown in this figure, system 900 comprises a motherboard or system-on-chip (SoC) 902 for mounting platform components. Motherboard or system-on-chip (SoC) 902 is a point-to-point (P2P) interconnect platform that includes a first processor 904 and a second processor 906 coupled via a point-to-point interconnect 970 such as an Ultra Path Interconnect (UPI). In other embodiments, the system 900 may be of another bus architecture, such as a multi-drop bus. Furthermore, each of processor 904 and processor 906 may be processor packages with multiple processor cores including core(s) 908 and core(s) 910, respectively as well as multiple registers, memories, or caches, such as, registers 912 and registers 914, respectively. While the system 900 is an example of a two-socket (2S) platform, other embodiments may include more than two sockets or one socket. For example, some embodiments may include a four-socket (4S) platform or an eight-socket (8S) platform. Each socket is a mount for a processor and may have a socket identifier. Note that the term platform refers to the motherboard with certain components mounted such as the processor 904 and chipset 932. Some platforms may include additional components and some platforms may only include sockets to mount the processors and/or the chipset. Furthermore, some platforms may not have sockets (e.g. SoC, or the like).

The processor 904 and processor 906 can be any of various commercially available processors, including without limitation an Intel® Celeron®, Core®, Core (2) Duo®, Itanium®, Pentium®, Xeon®, and XScale® processors; AMD® Athlon®, Duron® and Opteron® processors; ARM® application, embedded and secure processors; IBM® and Motorola® DragonBall® and PowerPC® processors; IBM and Sony® Cell processors; and similar processors. Dual microprocessors, multi-core processors, and other multi-processor architectures may also be employed as the processor 904 and/or processor 906. Additionally, the processor 904 need not be identical to processor 906.

Processor 904 includes an integrated memory controller (IMC) 920 and point-to-point (P2P) interface 924 and P2P interface 928. Similarly, the processor 906 includes an IMC 922 as well as P2P interface 926 and P2P interface 930. IMC 920 and IMC 922 couple the processors processor 904 and processor 906, respectively, to respective memories (e.g., memory 916 and memory 918). Memory 916 and memory 918 may be portions of the main memory (e.g., a dynamic random-access memory (DRAM)) for the platform such as double data rate type 3 (DDR3) or type 4 (DDR4) synchronous DRAM (SDRAM). In the present embodiment, the memories memory 916 and memory 918 locally attach to the respective processors (i.e., processor 904 and processor 906). In other embodiments, the main memory may couple with the processors via a bus and shared memory hub.

System 900 includes chipset 932 coupled to processor 904 and processor 906. Furthermore, chipset 932 can be coupled to storage device 950, for example, via an interface (I/F) 938. The I/F 938 may be, for example, a Peripheral Component Interconnect-enhanced (PCI-e). Storage device 950 can store instructions executable by circuitry of system 900 (e.g., processor 904, processor 906, GPU 948, ML accelerator 954, vision processing unit 956, or the like). For example, storage device 950 can store instructions for routine 400, routine 500, or the like.

Processor 904 couples to a chipset 932 via P2P interface 928 and P2P 934 while processor 906 couples to a chipset 932 via P2P interface 930 and P2P 936. Direct media interface (DMI) 976 and DMI 978 may couple the P2P interface 928 and the P2P 934 and the P2P interface 930 and P2P 936, respectively. DMI 976 and DMI 978 may be a high-speed interconnect that facilitates, e.g., eight Giga Transfers per second (GT/s) such as DMI 3.0. In other embodiments, the processor 904 and processor 906 may interconnect via a bus.

The chipset 932 may comprise a controller hub such as a platform controller hub (PCH). The chipset 932 may include a system clock to perform clocking functions and include interfaces for an I/O bus such as a universal serial bus (USB), peripheral component interconnects (PCIs), serial peripheral interconnects (SPIs), integrated interconnects (I2Cs), and the like, to facilitate connection of peripheral devices on the platform. In other embodiments, the chipset 932 may comprise more than one controller hub such as a chipset with a memory controller hub, a graphics controller hub, and an input/output (I/O) controller hub.

In the depicted example, chipset 932 couples with a trusted platform module (TPM) 944 and UEFI, BIOS, FLASH circuitry 946 via I/F 942. The TPM 944 is a dedicated microcontroller designed to secure hardware by integrating cryptographic keys into devices. The UEFI, BIOS, FLASH circuitry 946 may provide pre-boot code.

Furthermore, chipset 932 includes the I/F 938 to couple chipset 932 with a high-performance graphics engine, such as, graphics processing circuitry or a graphics processing unit (GPU) 948. In other embodiments, the system 900 may include a flexible display interface (FDI) (not shown) between the processor 904 and/or the processor 906 and the chipset 932. The FDI interconnects a graphics processor core in one or more of processor 904 and/or processor 906 with the chipset 932.

Additionally, ML accelerator 954 and/or vision processing unit 956 can be coupled to chipset 932 via I/F 938. ML accelerator 954 can be circuitry arranged to execute ML related operations (e.g., training, inference, etc.) for ML models. Likewise, vision processing unit 956 can be circuitry arranged to execute vision processing specific or related operations. In particular, ML accelerator 954 and/or vision processing unit 956 can be arranged to execute mathematical operations and/or operands useful for machine learning, neural network processing, artificial intelligence, vision processing, etc.

Various I/O devices 960 and display 952 couple to the bus 972, along with a bus bridge 958 which couples the bus 972 to a second bus 974 and an I/F 940 that connects the bus 972 with the chipset 932. In one embodiment, the second bus 974 may be a low pin count (LPC) bus. Various devices may couple to the second bus 974 including, for example, a keyboard 962, a mouse 964 and communication devices 966.

Furthermore, an audio I/O 968 may couple to second bus 974. Many of the I/O devices 960 and communication devices 966 may reside on the motherboard or system-on-chip (SoC) 902 while the keyboard 962 and the mouse 964 may be add-on peripherals. In other embodiments, some or all the I/O devices 960 and communication devices 966 are add-on peripherals and do not reside on the motherboard or system-on-chip (SoC) 902.

Embodiments of the present disclosure provide numerous advantages. For example, the invention reduces the number of inputs to and interactions with the robotic arthroplasty system required of the user during the surgical procedure. As such, the invention aids in the reduction in the time needed to complete the procedure and, additionally, reduces the opportunity for human-induced errors during the procedure. A machine learning model is trained on a dataset comprising data collected from a plurality of arthroplasty procedures such that the system is able to adapt configuration default settings of the robotic arthroplasty system to align default settings with the historical usage of the system by the user.

Although the present invention and its advantages have been described in detail, it should be understood that various changes, substitutions and alterations can be made herein without departing from the spirit and scope of the invention as defined by the appended claims. Moreover, the scope of the present application is not intended to be limited to particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the correspondent embodiments described herein may be utilized according to the present invention. Accordingly, the appended claims are intended to include within the scope, scenes, manufacture, compositions of matter, means, methods, or steps.

1. A system comprising:
a processor;
one or more machine learning models; and
memory storing software that, when executed by the processor, causes the system to:
receive, as input to the one or more machine learning models, information about an arthroplasty procedure to be performed;
generate, via the one or more machine learning models, configuration and default settings for the robotic arthroplasty system; and
send the configuration and default settings to the robotic arthroplasty system.

2. The system of claim 1, wherein the one or more machine learning models are trained to generate the configuration and default settings of the robotic arthroplasty system based on historical usage of one or more users of the robotic arthroplasty system.

3. The system of claim 2, wherein a training dataset for the one or more machine learning models includes particular bone types, bone features, bone dimensions or other anatomical features and structures from a plurality of arthroplasty procedures.

4. The method of claim 1, wherein the one or more machine learning models take as input one or more of an identification of the user, a type of procedure being performed and patient demographics.

5. The system of claim 4, wherein the configuration and default settings of the robotic arthroplasty system include one or more of implant position, a selection of views depicted in a graphical user interface of the system and an order in which the selection of views is displayed.

6. The system of claim 1, wherein the one or more machine learning models are trained to discriminate at least on a user-by-user basis, such that an input of different users results in generation of different configuration and default settings.

7. The system of claim 1, wherein the one or more machine learning models are updated on a per-procedure basis based on inputs received from a user during each procedure.

8. The system of claim 1, wherein the software further causes the system to:
receive, from the arthroplasty system, an indication of a value of at least one setting for a plurality of arthroplasty procedures, the plurality of arthroplasty procedures associated with a specific user of the robotic arthroplasty system; and
wherein the one or more machine learning models are trained based on the values of the at least one setting, to infer a default value of the at least one setting for a subsequent arthroplasty procedure associated with the specific user.

9. The system of claim 1, wherein the generated configuration and default settings comprise a user-specific configuration for the robotic arthroplasty system, the user-specific configuration including indications of a default value for at least one setting; and
wherein the software further causes the system to update a default configuration of the robotic arthroplasty system based on the user-specific configuration.

10. The system of claim 9, wherein the software further causes the system to:
record, during the plurality of arthroplasty procedures, an input to change a viewpoint displayed in a graphical user interface from a first viewpoint to a second viewpoint;
update the one of more machine learning models with the input to change a viewpoint; and
set, based on an output of the one of more machine learning models, for subsequent arthroplasty procedures, the second viewpoint as the value of a first one of the at least one setting.

11. The system of claim 9, wherein the software further causes the system to:
record, during the plurality of arthroplasty procedures, a plurality of demographic information for the patient;
update the one of more machine learning models with the input to change a viewpoint; and
set, based on an output of the one of more machine learning models, for subsequent arthroplasty procedures, the demographics information for the patient as the value of a third one of the at least one setting.

12. The system of claim 9, wherein the software further causes the system to:
record, during the plurality of arthroplasty procedures, an input to change an implant location from an initial location to an alternative location;
update the one of more machine learning models with the input to change a viewpoint; and
set, based on an output of the one of more machine learning models, for subsequent arthroplasty procedures, the alternative location as the value of a second one of the at least one setting.

13. The system of claim 1, wherein the software further causes the system to:
receive, from the arthroplasty system, an indication of a value of the at least one setting for a second plurality of arthroplasty procedures, the second plurality of arthroplasty procedures associated with a second user of the arthroplasty system; and
update the one or more machine learning models, based on the value of the at least one setting for the second plurality of arthroplasty procedures, to infer a default value of the at least one setting for a subsequent arthroplasty procedure associated with the second user.

14. The system of claim 13, wherein the user is a surgeon and the second user is a practice group.

15. The system of claim 1, wherein the system and the robotic arthroplasty system are integral.
